(19) United States
(12) Patent Application Publication
(10) Pub. No.: US 2004/0176779 A1
(43) Pub. Date: Sep. 9, 2004

(54) Targeting aid

(76) Inventors: Guido Casutt, Rickenbach-Sulz (CH); Mathias Heller, Raterschen (CH); Diego Frei, Kloten (CH)

Correspondence Address: CHRISTIE, PARKER & HALE, LLP, PO BOX 7068, PASADENA, CA, 91109-7068, US

(21) Appl. No.: 10/770,544

(22) Filed: Feb. 3, 2004

(30) Foreign Application Priority Data
Feb. 3, 2003 (EP) 03 002 321.2

(51) Int. Cl. A61B 17/28; A61B 17/88; A61F 2/46

(52) U.S. Cl. 606/102; 606/207

(57) ABSTRACT

The invention relates to a targeting aid for the spatial fixing of a target device relative to spinal vertebrae of a patient, comprising a clamping tool which is formed as a pair of forceps and which has at least two clamping jaws with which the clamping tool can be fixedly clamped to the spinal vertebrae while bridging at least two adjacent spinal vertebrae, and comprising at least one target base which is fixedly connected to the clamping tool and which is formed for coupling to a separate target device.

The invention relates to a targeting aid for the spatial fixing of a target base relative to the spinal vertebrae of a patient.

In particular in operations on the spinal column, the problem exists that the surgeon has to exactly position or place implants or fastening means such as pedicular screws in order to achieve a perfect result of the operation. This is difficult since the surgeon as a rule cannot determine the correct position or direction by just looking or feeling or by multiple attempts, but is dependent for this purpose on aids in the form of special target devices. Such target devices are known, for example, in the form of X-ray devices at the front of which a target unit having two reticles spaced along the optical axis can be fitted by means of a fixable pivot arm, with said reticles coinciding on a correct alignment and being able to be re-placed in the correctly aligned state by a guide bush through which, on the correct alignment of the target unit, a tool can be guided, for example an awl, with which a setting on point can be marked and a passage can be drilled for a pedicular screw.

A further problem exists with navigation systems for the immovable fastening of a reference coordinate system to a bone to be operated on. If it is a case of an individual spinal vertebra, such reference coordinate systems, which include, for example, a carrier body having three reflective spheres, are very sensitive with respect to their fastening.

What is problematic with these and other target devices are the difficulties in their correct alignment relative to the spinal column, or relative to the spinal vertebrae of the patient. The invention is designed to provide a remedy here.

It is therefore the object of the invention to provide a possibility to be able to correctly position a target device for the respective operational purpose in a simple and reliable manner.

This object is satisfied by the features of claim 1, and in particular in that the targeting aid has a clamping tool which is designed in the form of forceps and has at least two clamping jaws with which the clamping tool can be fixedly clamped to the spinal vertebrae while bridging at least two adjacent spinal vertebrae, and includes at least one target base which is fixedly connected to the clamping tool and which is designed for the coupling to a separate target device.

Clamping forceps are provided by the invention with which a target base can be spatially fixed relative to the spinal vertebrae in a very simple and nevertheless extremely reliable manner. A reference point is hereby provided by the target base whose position relative to the spinal vertebrae bridged by means of the clamping jaws is fixed in an unchangeable manner as long as the clamping forceps are fixedly clamped to the spinal vertebrae. By means of the target base spatially fixed in this manner, a separate target device can now be coupled which can be positioned and aligned in a clearly defined, and in particular reproducible manner, relative to the spinal vertebrae bridged by means of the clamping forceps.

Preferred embodiments of the invention are recited in the dependent claims, in the description and in the drawing.

The clamping forceps can have two levers which are rotatably connected to one another about an axis of rotation, which are each provided with an actuation section at the one side of the axis of rotation and support one of the clamping jaws at the other side of the axis of rotation.

Provision is furthermore preferably made for the levers for the securing of an established clamping state to be able to be fixed relative to one another.

The fixation, fixing or locking of the clamping forceps can take place such as is known from conventional surgical tools.

The levers can be designed such that, in the fixedly clamped state, the levers extend substantially parallel to one another at least in the region of the clamping jaws.

Provision can furthermore be made for the clamping jaws to extend substantially perpendicular to a plane set up by the two levers.

In a further preferred embodiment of the targeting aid in accordance with the invention, the one clamping jaw is fixedly secured to the respective lever and the other clamping jaw is rotatably secured to the respective lever. A further degree of freedom of movement is hereby advantageously provided which results in an improved alignability of the clamping jaws relative to the spinal vertebrae to be bridged.

Provision can furthermore be made for at least one clamping jaw to include a transverse support which extends perpendicular to a plane set up by the two levers and which supports at least two clamping members arranged spaced from one another and each having a clamping surface facing the other clamping jaw.

With an appropriately selected spacing of the clamping members, the clamping forceps in accordance with the invention can hereby be directly set onto the spinal vertebrae to be bridged, or onto the spines of the vertebrae, using the clamping members.

If, in accordance with a preferred embodiment, both clamping jaws include a respective transverse support provided with one or more clamping members, the clamping members can advantageously be arranged disposed opposite one another.

The clamping surfaces are formed, for example, in a circular manner in each case. Furthermore, the clamping surfaces can each be roughened or structured. A possibility for a structuring consists, for example, of forming a plurality of notches in each case in the clamping surfaces.

Provision can furthermore be made in accordance with the invention for the clamping members to be movably mounted at the transverse support. The clamping jaws can hereby be better matched to the spinal vertebrae to be bridged.

A respective joint can be provided between the clamping members and the transverse support. This can in particular be a ball joint.

The central spacing between two adjacent clamping members of a clamping jaw preferably corresponds to the central spacing of the spines of two adjacent spinal vertebrae in the lumbar region.

The central spacing between two adjacent clamping bodies of a clamping jaw can in particular lie in the range of approximately 32 mm±5 mm.

An advantage of this arrangement lies in the fact that in each case a rigid part of the clamping forceps—namely the clamping jaw in each case—is aligned in a defined manner with the pivotal clamping members, which are in particular formed in plate shape, at the spines of the vertebrae, when the oppositely disposed clamping members guided in the intended manner via the clamping forceps are pressed on from the other side. In this manner, the vertebrae are connected to one another via a rigid bridge.

The target base can project from the clamping forceps extending approximately parallel to the clamping jaws. The target base in particular projects approximately perpendicularly from one lever of the clamping forceps.

The invention furthermore relates to a target system comprising a targeting aid such as has been explained above and comprising at least one separate target device which can be coupled to the target base of the targeting aid.

The invention will be described in the following by way of example with reference to the drawing. There are shown:

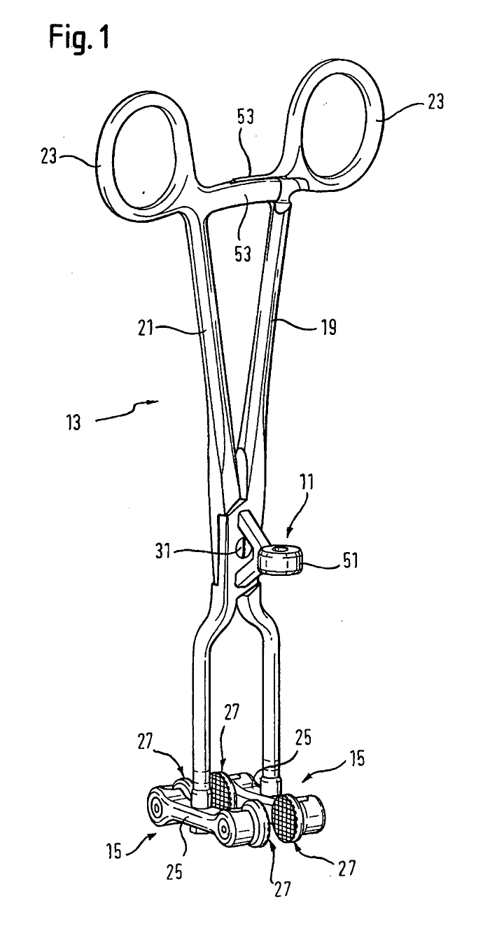

FIG. 1 a perspective view of a targeting aid in accordance with an embodiment of the invention;

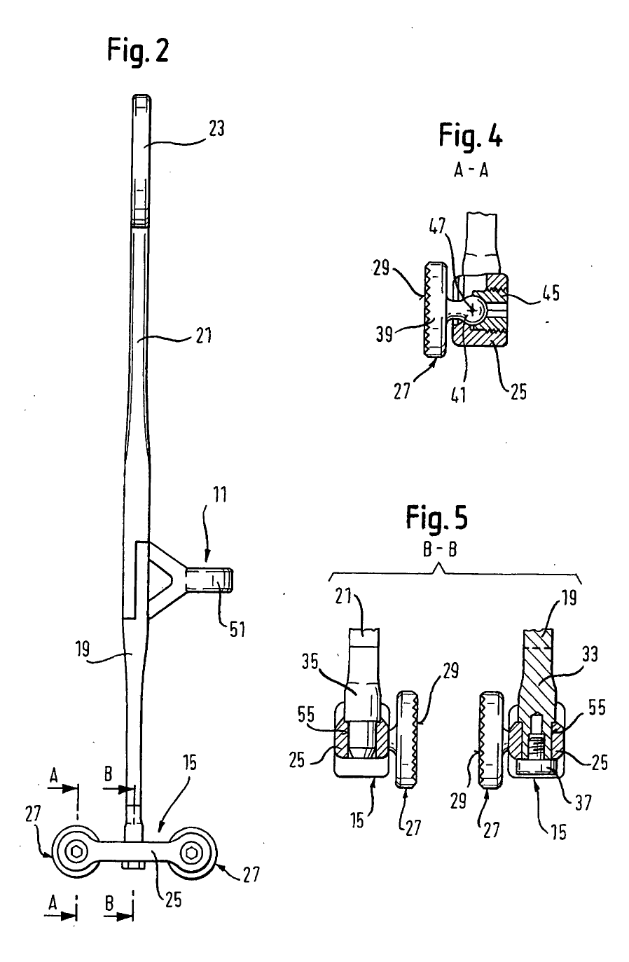

FIG. 2 the targeting aid of FIG. 1 in a side view;

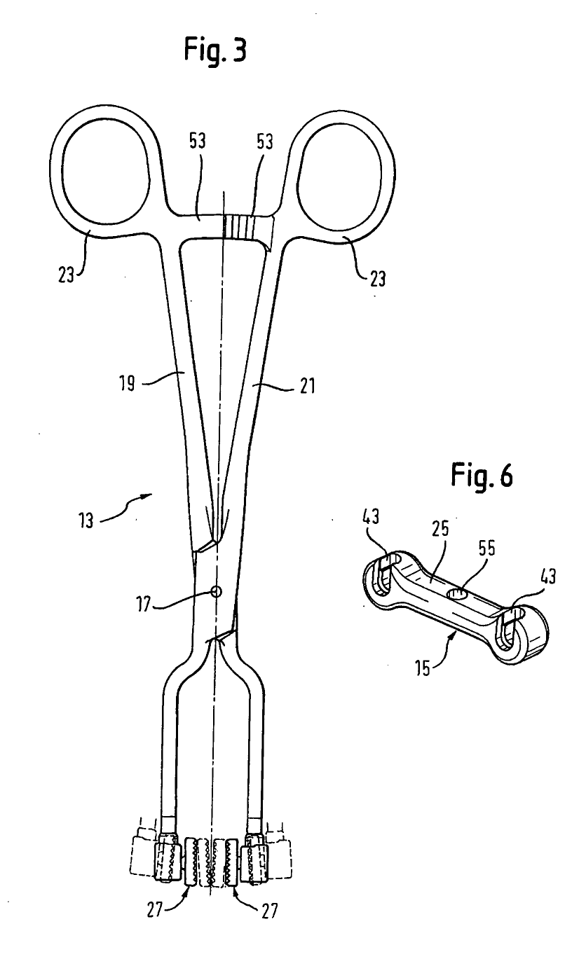

FIG. 3 the targeting aid of FIG. 1 in a plan view with clamping members located at different positions;

FIGS. 4 and 5 in each case, an enlarged detail of the targeting aid in accordance with the invention shown in section; and

FIG. 6 a perspective view of a transverse support of the targeting aid in accordance with the invention forming a component of a clamping jaw.

The targeting aid in accordance with the invention shown in FIGS. 1 to 6 includes clamping forceps 13 having two levers 19, 21 hinged to one another about an axis of rotation 17. The connection between the two levers 19, 21 takes place in the region of the axis of rotation 17 by a pivot screw 31 which has a supporting cylindrically designed section for the lever 19 and is screwed to the lever 21. Moreover, a rotational security is provided between the pivot screw 31 and the lever 21.

At free lever ends disposed opposite to actuation sections 23 of the levers 19, 21, a clamping jaw 15 is respectively provided having a bone-shaped straight transverse support 25 which supports in each case at its free end regions expanded perpendicular to its elongate extent a movably supported clamping member 27 which will be looked at in more detail in the following. The clamping members 27 of the levers 19, 21 are arranged opposite one another when the clamping forceps 13 are closed such that the clamping surfaces 29 (which will likewise be looked at in more detail in the following) of the oppositely disposed clamping members 27 face one another directly.

The attachment of the two transverse supports 25 to the free ends of the levers 19, 21 differs in that the one transverse support 25 (the left hand one in FIG. 5) is fixedly connected—e.g. by pressing in and/or welding—to the relevant lever 21 such that the lever 21 and the transverse support 25 form a rigid T, whereas the other transverse support 25 (the right hand one in FIG. 5) is attached in a rotatably mounted manner to the relevant lever 19, with the axis of rotation coinciding with the central axis of the lever 19 and thus extending perpendicular to the elongate extent of the transverse carrier 25.

The different attachment of the transverse supports 25 to the levers 19, 21 takes place by means of differently designed connection spigots 33, 35 which are fixedly connected at their one end to the free end of the respective lever 19, 21 and onto whose free end the respective transverse support 25 is placed which has, for this purpose, a centrally arranged bore 55. Whereas the connection between the transverse support 25 and the connection spigot 35 of the lever 21 is rotationally fixed, the transverse carrier 25 journalled on the connection spigot 33 of the lever 19 can be rotated relative to the connection spigot 33. The fastening of the transverse carrier 25 to the connection spigot 33 takes place by a fastening screw 37 which is screwed to the connection spigot 33.

The clamping members 27 each include a cylindrical clamping plate 39 having a circular clamping surface 29 as well as a bearing section 41 having a spherical bearing head and projecting perpendicularly from the side of the clamping plate 39 disposed opposite to the clamping surface 29.

The transverse carriers 25 each have a receiving space 43 (cf. in particular FIG. 6) in their extended end regions for the bearing section 41 of the respective clamping member 27. The spherical bearing head can be inserted (from above in FIG. 6) into this receiving space 43.

A closing screw 45, which can be screwed into the receiving space 43 from the rear, serves as the support for the bearing section 41 of the clamping member 27. For this purpose, the closing screw 45 is formed at its side facing the clamping member 27 as a cup bearing which forms a ball joint together with the bearing section 41. The clamping member 27 is hereby freely movable in all directions about a pivot point 47 at a fixed position with respect to the transverse carrier 25, whereby different orientations of the clamping surface 29 relative to the transverse carrier 25 are possible. At the same time, the clamping member 27 is secured by the closing screw 45 against unwanted translation movements relative to the transverse support 25.

The clamping surfaces 29 are roughened or structured—for example by the provision of a plurality of notches arranged at right angles to one another—in order to ensure a secure and locally stable fixed clamping of the clamping forceps 13 in accordance with the invention to the spinal vertebrae of the patient.

The movable support of the clamping members 27, together with the rotatability of the one transverse support 25, allows any desired relative positions of the four clamping surfaces and thus ensures an optimum matching of the clamping forceps 13 to the respective spinal vertebrae to be bridged.

Different positions of the clamping jaws 15 including the transverse sup-ports 25 and the clamping members 27 are indicated schematically in FIG. 3.

A target base 11 of the targeting aid in accordance with the invention serving for the coupling to a separate target device (not shown)—for example to a target device such as is described in the introductory part—is fixedly connected to the clamping forceps 13, for example by welding. In the embodiment shown, the target base 11 projects in a perpendicular manner from the one lever 19 and thus extends parallel to the clamping jaws 15. The target base 11 has a Y shape, with the two Y limbs being welded at their free ends to the lever 19 and the straight Y section being formed by the annular section 51 which forms the actual base and via which the coupling to the separate target device takes place.

The design of the target base 11, or of the annular section 51, can generally be of any kind and is matched to the target device to be coupled.

To fix the target base 11 spatially relative to spinal vertebrae of a patient, the clamping bodies 27 of the clamping jaws 15 of the clamping forceps 13 are set in the open state at the spines of two adjacent spinal vertebrae. The central spacing of the clamping bodies 27 of each transverse support 25 is selected in accordance with the central spacing of the spines of the vertebrae of the respective patient and amounts in the embodiment shown to 32 mm.

The fixing of the clamping forceps 13 to the spinal vertebrae takes place by pressing together the two actuation sections 23. Latching arms 53 of the two levers 19, 21, which cooperate in the manner of a ratched and which are biased towards one another due to their orientation relative to the levers 19, 21, secure the intermediate and end positions achieved in each case by being pressed together on the closing of the clamping forceps 13 and prevent a release of the clamping when the clamping forceps 13 are released.

The latching arms 53 are brought out of engagement again, whereby the clamping can again be released, by a slight moving apart of the two levers 19, 21 in the region of the actuation sections 23 perpendicular to the plane set up by them.

For the better monitoring of the pedicular screws set using the targeting aid in accordance with the invention, or using the clamping forceps, by means of image amplifiers, in particular in the form of X-ray devices, the clamping pliers can be made at least partly of plastic reinforced with carbon fibres.

### REFERENCY NUMERAL LIST

11 target base

13 clamping tool, clamping forceps

15 clamping jaw

17 axis of rotation

19 lever

21 lever

23 actuation section

25 transverse support

27 clamping member

29 clamping surface

31 pivot screw

33 connection spigot (rotatable)

35 connection spigot (fixed)

37 fastening screw

39 clamping plate

41 support section

43 receiving space

45 closing screw

47 pivot point

51 annular section of the target base

53 latching arm

55 bore

1. A targeting aid for the spatial fixing of a target device relative to spinal vertebrae of a patient, comprising
a clamping tool (13) which is formed as a pair of forceps and which has at least two clamping jaws (15) with which the clamping tool (13) can be fixedly clamped to the spinal vertebrae while bridging at least two adjacent spinal vertebrae; and
at least one target base (11) which is fixedly connected to the clamping tool (13) and is formed for the coupling to a separate target device.

2. A targeting aid in accordance with claim 1, characterised in that the clamping forceps (13) have two levers (19, 21) which are rotatably connected to one another about an axis of rotation (17) and which are provided with an actuation section (23) at the one respective side of the axis of rotation (17) and support one of the clamping jaws (15) at the other side of the axis of rotation (17).

3. A targeting aid in accordance with claim 2, characterised in that the levers (19, 21) can be fixed relative to one another for the securing of an established clamping state.

4. A targeting aid in accordance with claim 2, characterised in that, in the fixedly clamped state, the levers (19, 21) extend substantially parallel to one another at least in the region of the clamping jaws (15).

5. A targeting aid in accordance with claim 2, characterised in that the clamping jaws (15) extend substantially perpendicular to a plane set up by the two levers (19, 21).

6. A targeting aid in accordance with claim 1, characterised in that the one clamping jaw (15) is rotatably secured to the clamping forceps (13) and the other clamping jaw (15) is fixedly secured to the clamping forceps (13).

7. A targeting aid in accordance with claim 2, characterised in that at least one clamping jaw (15) includes a transverse support (25) which extends substantially perpendicular to a plane set up by the two levers (19, 21) and which supports at least two clamping members (27) arranged spaced apart from one another and each having a clamping surface (29) facing the other clamping jaw (15).

8. A targeting aid in accordance with claim 7, characterised in that the clamping surfaces (29) are each circular.

9. A targeting aid in accordance with claim 7, characterised in that the clamping surfaces (29) are each roughened or structured.

10. A targeting aid in accordance with claim 7, characterised in that the clamping surfaces (29) each have a plurality of notches running through them.

11. A targeting aid in accordance with claim 7, characterised in that the clamping members (27) are each movably supported at the transverse support (25).

12. A targeting aid in accordance with claim 7, characterised in that a joint, in particular a ball joint, is respectively provided between the clamping members (27) and the transverse support (25).

13. A targeting aid in accordance with claim 7, characterised in that the spacing between adjacent clamping members (27) of a clamping jaw (15) corresponds to the central spacing of the spines of two adjacent spinal vertebrae in the lumbar region.

14. A targeting aid in accordance with claim 7, characterised in that the spacing between two adjacent clamping members (27) of a clamping jaw (15) lies in the range from 27 mm to 37 mm.

15. A targeting aid in accordance with claim 1, characterised in that the target base (11) projects from the clamping forceps (13) extending approximately parallel to the clamping jaws (15).

16. A targeting aid in accordance with claim 1, characterised in that the target base (1) projects approximately perpendicularly from one lever (19) of the clamping pliers (13).

17. A targeting aid in accordance with claim 2, characterised in that the target base (11) is fixedly connected to one of the levers (19, 21), in particular in the region of the axis of rotation (17) of the two levers (19, 21).

18. A targeting aid in accordance with claim 1, characterised in that it is made at least partly from plastic reinforced with carbon fibres.

19. A target system comprising a targeting aid in accordance with claim 1 and comprising at least one separate target device which can be coupled to the target base (11) of the targeting aid.
